(19) United States
(12) Patent Application Publication
(10) Pub. No.: US 2012/0018529 A1
(43) Pub. Date: Jan. 26, 2012

(54) Air Freshener Device

(76) Inventors: Frederick A. Gammon, South Orange, NJ (US); Michael Koulermos, New Albany, OH (US); Anthony R. Zembrodt, Covington, KY (US)

(21) Appl. No.: 13/185,928

(22) Filed: Jul. 19, 2011

Related U.S. Application Data
(60) Provisional application No. 61/365,948, filed on Jul. 20, 2010.

(51) Int. Cl. A61L 9/04

(52) U.S. Cl. 239/6; 239/44; 239/34

(57) ABSTRACT

An air freshener device includes a base unit and a fragrance cartridge. The base unit includes an electrical connector, a heating element, a pair of interior sidewalls, an interior end wall, and a first shaped feature. The pair of interior sidewalls and the interior end wall extend substantially vertically from the floor portion and cooperate to at least partially define a receptacle. The first shaped feature is associated with one of the floor portion, the interior end wall, and one of the pair of interior sidewalls. The fragrance cartridge includes an absorbent pad, a shell body, and a second shaped feature. The shell body substantially encompasses and supports the absorbent pad and includes a shell wall. The second shaped feature is associated with the shell wall and is configured for mated interaction with the first shaped feature of the base unit.

### CROSS-REFERENCE TO RELATED APPLICATION

This application claims priority of U.S. provisional patent application Ser. No. 61/365,948, filed Jul. 20, 2010 which is hereby incorporated by reference herein in its entirety.

### TECHNICAL FIELD

An air freshener device includes a base unit and a fragrance cartridge. The base unit includes a heating element for heating the fragrance cartridge.

### BACKGROUND

A USB-type air freshener device includes a heating element that is configured to heat and volatize a fragrance composition.

### SUMMARY

In accordance with one embodiment, an air freshener device comprises a base unit and a fragrance cartridge. The base unit comprises a first end and a second end. The first end comprises an electrical connector. The second end comprises a floor portion, a heating element, a pair of interior sidewalls, an interior end wall, and a first shaped feature. The heating element is coupled with the floor portion and is electrically coupled with the electrical connector. The pair of interior sidewalls extends substantially vertically from the floor portion and on opposite sides of the heating element. The interior end wall extends substantially vertically from the floor portion and extends between the pair of interior sidewalls. The interior end wall and the pair of interior sidewalls cooperate to at least partially define a receptacle. The first shaped feature is associated with one of the floor portion, the interior end wall, and one of the pair of interior sidewalls. The fragrance cartridge is at least partially disposed within the receptacle and overlies the heating element. The fragrance cartridge comprises an absorbent pad, a shell body, and a second shaped feature. The absorbent pad is configured to selectively retain a fragrance composition. The shell body substantially encompasses and supports the absorbent pad and comprises a shell wall. The second shaped feature is associated with the shell wall and is configured for mated interaction with the first shaped feature of the base unit. The fragrance cartridge is configured for selective installation within the receptacle.

In accordance with another embodiment, a method for operating an air freshener device is provided. The air freshener device comprises a base unit and a fragrance cartridge. The base unit defines a receptacle and includes an electrical connector and a heating element electrically coupled with the electrical connector. The fragrance cartridge is configured for selective installation into the receptacle. The method comprises connecting the electrical connector with a power source such that electrical power is transmitted to the electrical connector. The method further comprises deactivating the heating element when the fragrance cartridge is not installed in the receptacle; and controlling activation of the heating element when the fragrance cartridge is installed into the receptacle.

In accordance with another embodiment, an air freshener device comprises a base unit and a fragrance cartridge. The base unit comprises a front end, a rear end, and an electrical switch. The front end comprises a USB plug. The rear end comprises a floor portion, a heating element, a pair of interior sidewalls, an interior end wall, and a shaped receptacle. The heating element is coupled with the floor portion and is electrically coupled with the USB plug. The pair of interior sidewalls extends substantially vertically from the floor portion and on opposite sides of the heating element. The interior end wall extends substantially vertically from the floor portion and extends between the pair of interior sidewalls. The interior end wall and the pair of interior sidewalls cooperate to at least partially define a receptacle. The shaped receptacle is defined by the interior end wall. The electrical switch is in electrical communication with each of the USB plug and the heating element. The electrical switch is associated with the shaped receptacle. The fragrance cartridge is at least partially disposed within the receptacle and overlies the heating element. The fragrance cartridge comprises an absorbent pad, a shell body, and a shaped member. The absorbent pad is configured to selectively retain a fragrance composition. The shell body substantially encompasses and supports the absorbent pad and comprises a shell wall. The shaped member extends from the shell wall and is configured for mated interaction with the shaped receptacle of the base unit. The fragrance cartridge is configured for selective installation within the receptacle. The electrical switch is configured for operation when the shaped member is mated with the shaped receptacle. When the electrical switch is operated, the heating element is selectively activated according to a predetermined timing arrangement.

### BRIEF DESCRIPTION OF THE DRAWINGS

Various embodiments will become better understood with regard to the following description, appended claims and accompanying drawings wherein:

FIG. 1 is a right front perspective exploded view depicting an air freshener device including a base unit, a fragrance cartridge, and a plug cover, in accordance with one embodiment;

FIG. 2 is a top plan view depicting the base unit of FIG. 1;

FIG. 3 is a left rear perspective view depicting the fragrance cartridge of FIG. 1;

FIG. 4 is a right front perspective view depicting the air freshener device of FIG. 1 with the fragrance cartridge installed in the base unit;

FIG. 5 is a left rear perspective view depicting the base unit of FIG. 1;

FIG. 6 is a rear plan view depicting the base unit of FIG. 1;

FIG. 7 is a right front perspective view depicting a fragrance cartridge according to another embodiment;

FIG. 8 is a right front perspective view depicting a fragrance cartridge according to yet another embodiment; and

FIG. 9 a left rear perspective view depicting a base unit according to another embodiment.

### DETAILED DESCRIPTION

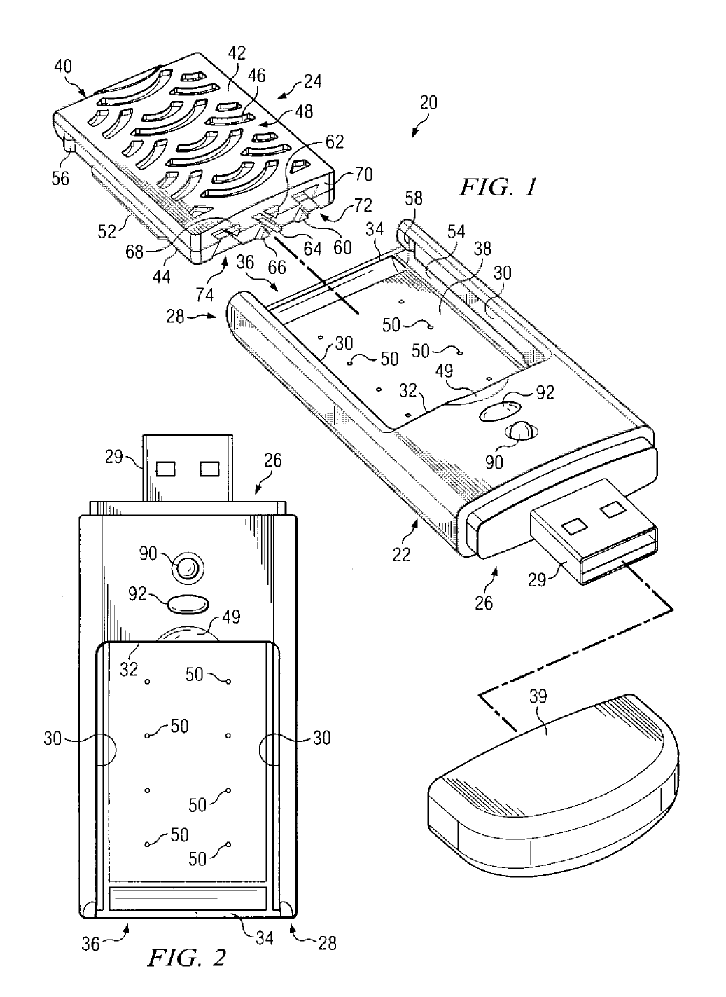

Selective embodiments are herein after described in detail in connection with the views and examples of FIGS. 1-9, wherein like numbers indicate the same or corresponding elements throughout the views. An air freshener device 20 is illustrated in FIG. 1 and can include a base unit 22 and a fragrance cartridge 24. The base unit 22 can include a front end 26 and a rear end 28. The base unit 22 can include an electrical connector 29 that extends from the front end 26. The rear end 28 of the base unit 22 can include a pair of interior sidewalls 30 and a front wall 32 each of which extends substantially vertically from a floor portion 34. The interior sidewalls 30, the front wall 32, and the floor portion 34 can cooperate to define a receptacle 36. A heating element 38 can be coupled with the floor portion 34 such that the heating element 38 is disposed within the receptacle 36 and resides between the interior sidewalls 30.

The heating element 38 can be in electrical communication with the electrical connector 29. When the electrical connector 29 is plugged into a power source (not shown), electrical power from the power source can be communicated to the heating element 38 to energize the heating element 38 and produce heat. In one embodiment, as illustrated in FIGS. 1 and 2, the electrical connector 29 can comprise a Universal Serial Bus (“USB”) type plug. The air freshener device 20 can accordingly be plugged into and powered from a USB port such as may be provided on a computer, personal digital assistant, cellular phone, USB-type wall charger, or incorporated within a vehicle, such as an automobile, for example. The electrical connector 29 can accordingly comprise a type-A USB plug, as shown in FIGS. 1 and 2, or any of a variety of suitable alternative USB plug arrangements (e.g., a type-B USB plug, a mini-A USB plug, a mini-B USB plug, a micro-AB USB plug, a micro-B USB plug, or a SuperSpeed USB Plug). In one embodiment, the electrical connector 29 can be plugged into a USB-type adapter that facilitates powering of the base unit 22 from a vehicle's 12-volt accessory power output (e.g., cigarette lighter). It will be appreciated, however, that the electrical connector 29 can be any of a variety of electrical connectors that facilitate external powering of the air freshener device 20 such as a conventional two-prong AC plug or a conventional three-prong AC plug. In some arrangements, when AC power is used to power the air freshener device 20, it will be appreciated that the base unit 22 can include a step down voltage converter that can convert AC power into an appropriate DC voltage for powering the heating element 38. It will also be appreciated that in lieu of an electrical connector, the air freshener device 20 can be powered by on-board batteries. The air freshener device 20 can include a plug cover 39 that can selectively cover the electrical connector 29.

In one embodiment, as illustrated in FIG. 1, the fragrance cartridge 24 can include a shell body 40 having an upper shell portion 42 and a lower shell portion 44. The upper and lower shell portions 42, 44 can cooperate to support an absorbent pad, generally indicated at 46, that retains a fragrance composition. The absorbent pad 46 can be configured in a variety of suitable alternative arrangements. In one embodiment, the absorbent pad 46 can be a cellulose pad that is infused with a liquid fragrance composition. The cellulose pad can comprise cardboard, recycled newspaper, wood pulp, or any of a variety of suitable alternative absorbent cellulose-type material. In other embodiments, the absorbent pad 46 can comprise cotton, porous plastic, ceramic or any of a variety of other suitable absorbent materials that can be infused with a fragrance composition and heated to release a fragrance. It will be appreciated that a fragrance cartridge can be provided in any of a variety of suitable alternative arrangements, such as in a gel pack-type arrangement.

The upper and lower shell portions 42, 44 can be releasably coupled together such that they move relative to one another. In one embodiment, the upper and lower shell portions 42, 44 can be provided in a two-piece arrangement. In such an embodiment, as illustrated in FIG. 1, the upper and lower shell portions 42, 44 can include internal securing tabs (not shown) that releasably attach the upper and lower shell portions 42, 44 together. In another embodiment, the upper and lower shell portions 42, 44 can be configured in a clamshell-type arrangement. In such an arrangement, the upper and lower shell portions 42, 44 are coupled together at one end by a pair of hinges (not shown). During assembly of the fragrance cartridge 24, the pair of hinges can secure the upper and lower shells portions 42, 44 together to reduce the risk of the upper or lower shell portions 42, 44 being misplaced or misassembled. In another embodiment, the upper and lower shell portions 42, 44 can be permanently affixed together. In one embodiment, the shell body 40 can be constructed of a thermoplastic material such as may be formed through a molding process (e.g., injection molding or blow molding) or an extrusion process, for example. In other embodiments, the shell body 40 can be constructed from any of a variety of other suitable alternative materials.

The fragrance cartridge 24 can be installed in the receptacle 36 such that the fragrance cartridge 24 overlies the heating element 38. In one embodiment, as illustrated in FIGS. 1 and 2, a notch 49 can be defined adjacent the front wall 32 to assist with the effective removal of the fragrance cartridge 24 by a user. The heating element 38 can be energized to facilitate heating of the absorbent pad 46. When the absorbent pad 46 is heated by the heating element 38, the fragrance composition retained by the absorbent pad 46 can be volatilized and thus released into the surrounding environment, producing a noticeable scent. The upper and lower shell portions 42, 44 can each define a plurality of vent holes (e.g., 48) that facilitate more effective heating of the absorbent pad 46 and thus more effective dispensation of a scent from the fragrance cartridge 24. For example, when the fragrance cartridge 24 is provided within the receptacle 36, heat from the heating element 38 can permeate the absorbent pad 46 through the vent holes (not shown) of the lower shell portion 44 to facilitate emission of fragrance through the vent holes 48 of the upper shell portion 42. It will be appreciated that the arrangement and configuration of vent holes can vary to provide the most effective release of fragrant scent.

In one embodiment, as illustrated in FIG. 1, the heating element 38 can cover almost the entire floor portion 34 such that the heating element 38 can substantially underlie the fragrance cartridge 24. In another embodiment, the heating element 38 can cover the entire floor portion 34. In one embodiment, the heating element 38 can include an array of heating resistors (e.g., thick film-chip transistors) or any of a variety of suitable alternative components that facilitate generation of thermal energy. In one embodiment, as illustrated in FIG. 1, the heating element 38 can comprise a pad-type heater that defines a plurality of through holes 50 that permit heat generated from the heating element 38 to more effectively reach the fragrance cartridge 24.

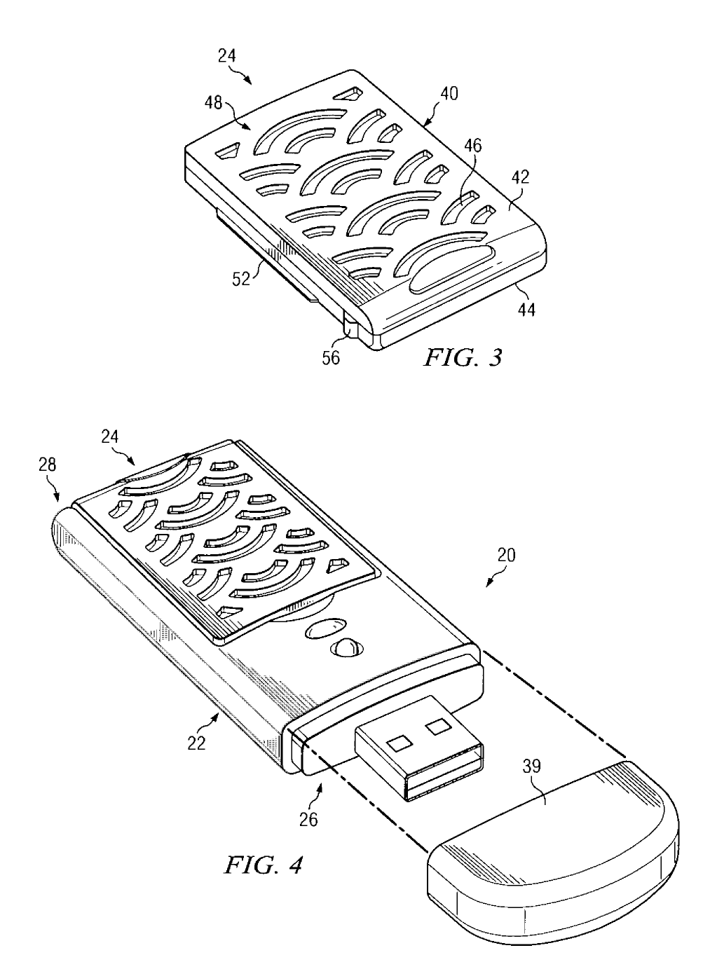

In one embodiment, the fragrance cartridge 24 can be installed in the receptacle 36 by sliding the fragrance cartridge 24 into the receptacle 36 from the rear of the base unit 22. As illustrated in FIGS. 1 and 3, the fragrance cartridge 24 can include a pair of guide fins 52 located on opposite sides of the shell body 40. When the fragrance cartridge 24 is slid into the receptacle 36, each of the guide fins 52 can project into a slot (e.g., 54) defined by each of the interior sidewalls 30. The guide fins 52 can thus facilitate guidance of the fragrance cartridge 24 into a fully installed position (shown in FIG. 4). Once the fragrance cartridge 24 is in the fully installed position, the guide fins 52 can facilitate retention of the fragrance cartridge 24 within the receptacle 36 (e.g., preventing the fragrance cartridge 24 from moving upwardly and being inadvertently separated from the base unit 22). As illustrated in FIGS. 1 and 3, the fragrance cartridge 24 can also include a pair of locking projections 56 located on opposite sides of the shell body 40. When the fragrance cartridge 24 is slid into the receptacle 36, the locking projections 56 can engage locking recesses (e.g., 58) defined by each of the interior sidewalls 30 utilizing an interference-type fit to facilitate securement of the fragrance cartridge 24 within the receptacle 36.

In one embodiment, the interior sidewalls 30 can each define a contoured inner surface (not shown) that corresponds with respective contoured sides (not shown) of the fragrance cartridge 24. When the fragrance cartridge 24 is being installed into the receptacle 36, the contoured inner surfaces of the interior sidewalls 30 can interact with the contoured sides of the fragrance cartridge 24 to guide the fragrance cartridge 24 into position over the heating element 38. Once the fragrance cartridge 24 is fully installed in the base unit 22, the interaction between the contoured inner surfaces of the interior sidewalls 30 and the contoured sides of the fragrance cartridge 24 can facilitate retention of the fragrance cartridge 24 within the receptacle 36. It will be appreciated that the contoured inner surfaces of the interior sidewalls 30 and the contoured sides of the fragrance cartridge 24 can be provided in lieu of or in addition to the guide fins 52 and/or the locking projections 56.

The fragrance cartridge 24 can additionally include a pushbutton (not shown). When the fragrance cartridge 24 is fully installed in the receptacle 36, the pushbutton can facilitate selective retention of the fragrance cartridge 24 with respect to the receptacle 36. The pushbutton can accordingly be depressed to facilitate selective release of the fragrance cartridge 24 from the receptacle 36. It will be appreciated that the fragrance cartridge 24 can include any of a variety of additional or alternative features that facilitate effective retention of the fragrance cartridge 24 within the receptacle 36.

In one embodiment, the fragrance cartridge 24 can be a single-use, disposable-type cartridge. In such an embodiment, once the fragrance cartridge 24 is no longer effective for use (e.g., because the fragrance composition has been consumed, destroyed, or because a user no longer appreciates the scent of the fragrance), the fragrance cartridge 24 can be removed from the receptacle 36 and replaced with a new fragrance cartridge. It will be appreciated that when a user grasps the fragrance cartridge 24 (e.g., either during the installation or removal of the fragrance cartridge), the shell body 40 can serve as a barrier to the absorbent pad 46 to prevent the user from directly contacting the absorbent pad 46 thereby reducing the risk that a fragrance composition contacts the user which could irritate the user's skin or be introduced into the user's eyes. The shell body 40 can also prevent the fragrance composition from being contacted by adjacent objects (e.g., a nearby computer) and/or underlying surfaces such as when the fragrance cartridge 24 is removed from the base unit 22 and laid upon a surface.

It will be appreciated that in some embodiments, the fragrance cartridge 24 might be configured as a multi-use, reusable-type unit. In such an embodiment, the absorbent pad 46 can comprise a durable absorbent material. A liquid fragrance composition can be dispensed onto the absorbent pad 46 through the vent holes (e.g., 48) in the upper and/or lower shell portions 42, 44. Once the liquid fragrance is consumed, additional liquid fragrance composition can be dispensed onto the absorbent pad 46 without requiring removal and/or replacement of the fragrance cartridge 24.

The base unit 22 and fragrance cartridge 24 can include respective shaped features that are configured for respective mated interaction when the fragrance cartridge 24 is installed within the receptacle 36. In one embodiment, as illustrated in FIG. 1, the fragrance cartridge 24 can include a left shaped cartridge member 60, three central shaped cartridge members 62, 64, 66, and a right shaped cartridge member 68 that each protrude from an end wall 70. The left and right shaped cartridge members 60, 68 are shown to be substantially triangularly shaped. The central shaped cartridge members 62, 64, 66 are shown to cooperate to form a substantially x-shaped feature. The fragrance cartridge 24 can also include left and right shaped cartridge receptacles 72, 74 that are defined by the end wall 70 and are shown to be substantially y-shaped. It will be appreciated by those having skill in the art that cartridge members and cartridge receptacles can be any of a variety of suitable alternative shapes or designs.

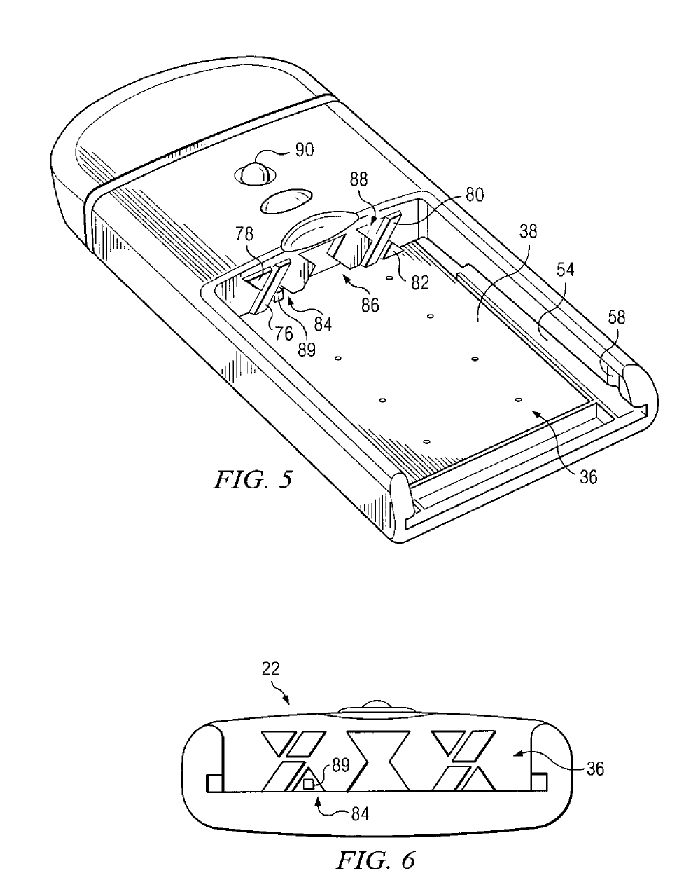

As illustrated in FIG. 5, the base unit 22 can include two left shaped base members 76, 78 and two right shaped base members 80, 82 that each protrude from the front wall 32. The base unit 22 can also include a left shaped base receptacle 84, a central shaped base receptacle 86, and a right shaped base receptacle 88 that are each defined by the front wall 32. The shape of the left and right shaped cartridge receptacles 72, 74 (e.g., y-shaped) can be similar to the overall shape of two left shaped base members 76, 78 and the two right shaped base members 80, 82, respectively. The shape of the left and right shaped base receptacles 84, 88 (e.g., triangularly shaped) can be similar to the shape of the left and right shaped cartridge members 60, 68, respectively. The shape of the central shaped base receptacle 86 (e.g., x-shaped) can be similar to the overall shape of the three central shaped cartridge members 62, 64, 66. It will be appreciated by those having skill in the art that base members and base receptacles can be any of a variety of suitable alternative shapes or designs.

When the fragrance cartridge 24 is fully installed in the receptacle 36, as illustrated in FIG. 4, the base unit 22 and the fragrance cartridge 24 can mate with each other. For example, the left shaped cartridge member 60 can be inserted into and can mate with the left shaped base receptacle 84, the three central shaped cartridge members, 62, 64, 66 can be inserted into and can mate with the central shaped base receptacle 86, the right shaped cartridge member 68 can be inserted into and can mate with the right shaped base receptacle 88, the left shaped base members 76, 78 can be inserted into and can mate with the left shaped cartridge receptacle 72, and the right shaped base members 80, 82 can be inserted into and can mate with the right shaped cartridge receptacle 74. The mated interaction between the shaped members (e.g., 60, 62, 64, 66, 68, 76, 78, 80, 82) and the respective shaped receptacles (e.g., 72, 74, 84, 86, 88) can demonstrate (e.g., to a user) that the fragrance cartridge 24 has been properly installed.

It will be appreciated that shaped members and/or shaped receptacle of a fragrance cartridge and/or base unit can be provided at any of a variety of suitable locations. For example, in an alternative embodiment, the fragrance cartridge 24 can include a shaped member that extends from a bottom wall and matedly interacts with a shaped receptacle defined by the floor portion 34 when the fragrance cartridge 24 is installed in the receptacle 36. In another embodiment, the fragrance cartridge 24 can include a shaped member that extends from a sidewall and matedly interacts with a shaped receptacle defined by one of the interior sidewalls 30 when the fragrance cartridge 24 is installed in the receptacle 36.

As illustrated in FIGS. 1 and 5, the shaped members (e.g., 60, 62, 64, 66, 68, 76, 78, 80, 82) and the shaped receptacles (e.g., 72, 74, 84, 86, 88) can be arranged to permit installation of the fragrance cartridge 24 within the receptacle 36 in an upright position (as shown in FIG. 1 and described above) and in an upside-down orientation (not shown). When the fragrance cartridge 24 is installed in the upside-down orientation, the left shaped cartridge member 60 can be inserted into and can mate with the right shaped base receptacle 88, the central shaped cartridge members, 62, 64, 66 can be inserted into and can mate with the central shaped base receptacle 86, the right shaped cartridge member 68 can be inserted into and can mate with the left shaped base receptacle 84, the left shaped base members 76, 78 can be inserted into and can mate with the right shaped cartridge receptacle 74, and the right shaped base members 80, 82 can be inserted into and can mate with the left shaped cartridge receptacle 72.

The air freshener device 20 can be configured to operate in different modes based upon the orientation of the fragrance cartridge 24 within the receptacle 36. In one embodiment, the air freshener device 20 can be configured to operate at different temperatures. For example, in one embodiment, when the fragrance cartridge 24 is installed in the upright position, the heating element 38 can heat the fragrance cartridge 24 at a high temperature (e.g., about 180° F.) and when the fragrance cartridge 24 is installed in the upside-down position (not shown), the heating element 38 can heat the fragrance cartridge 24 at a low temperature (e.g., about 120° F.). In another embodiment, the air freshener device 20 can be configured to operate at different cycle times. For example, in another embodiment, when the fragrance cartridge 24 is installed in the upright position, the heating element 38 can selectively heat the fragrance cartridge 24 at a particular interval of time (e.g., about every 30 minutes) and when the fragrance cartridge 24 is installed in the upside-down position (not shown), the heating element 38 can selectively heat the fragrance cartridge 24 at a different interval of time (e.g., about every 60 minutes). It will be appreciated that the air freshener device 20 can be configured to operate in any of a variety of suitable alternative modes based upon the orientation of the fragrance cartridge 24.

The shaped members and the shaped receptacles can be configured in any of a variety of arrangements that facilitate selective activation of the air freshener device 20 in different modes. For example, the left and right shaped base receptacles 84, 88 can include a magnetically-actuated contactor. The left shaped cartridge member 60 can include a magnet that can actuate either of the contactors depending upon the orientation of the fragrance cartridge 24 to facilitate activation of the air freshener device 20 in different modes.

The shaped members and the shaped receptacles can function as a lock and key arrangement between the base unit 22 and the fragrance cartridge 24. In particular, only fragrance cartridges that have corresponding shaped cartridge members and shaped cartridge receptacles that match the shaped base members and shaped base receptacles of the base unit 22 can be installed properly into the receptacle 36. In one embodiment, this lock and key arrangement can prevent certain fragrance cartridges from being installed in the receptacle 36 that might malfunction or cause the air freshener device 20 to malfunction. In one embodiment, only fragrance cartridges that are compatible with the base unit 22 (e.g., compatible fragrance cartridges) are provided with a shaped cartridge members and/or shaped cartridge receptacles that match the shaped base members and/or shaped base receptacles. All other fragrance cartridges that are not effectively compatible with the base unit 22 (e.g., non-compatible fragrance cartridges) can be provided with shaped cartridge members and/or shaped cartridge receptacles that are different from the shaped base member and/or the shaped base receptacle or alternatively provided without any shaped cartridge receptacles. If a user attempts to install a non-compatible fragrance cartridge into the base unit 22, the non-conforming fragrance cartridge will not properly interact with the shaped base members and shaped base receptacles thus demonstrating to the user that the non-compatible fragrance cartridge is not approved for use with the base unit 22. It will be appreciated that a fragrance cartridge might be deemed incompatible with an air fragrance device due to the shape of the fragrance cartridge, the size of the fragrance cartridge, non-compliance of the fragrance cartridge in meeting certain operational requirements (e.g., a knock-off fragrance cartridge), or the like. It will be also be appreciated that the shaped members and the shaped receptacles of the base unit 22 and the fragrance cartridge 24 can facilitate more effective and consistent installation of the fragrance cartridge 24 within the receptacle 36.

The base unit 22 can be configured to disable the heating element 38 until the fragrance cartridge 24 is properly installed within the receptacle 36 (e.g., with the shaped members and the shaped receptacles mated together). In one embodiment, the base unit 22 can include an electrical switch that is operable to facilitate selective disconnection of the electrical connector 29 from the heating element 38. The electrical switch can be electrically coupled with each of the electrical connector 29 and the heating element 38 such that the electrical connector 29 is disconnected from the heating element 38 non-actuation when the electrical switch is not operated. In one embodiment, as illustrated in FIGS. 5 and 6, the electrical switch can comprise a push-to-make type switch (e.g., a normally-open switch) having a contact finger 89 that extends from the left shaped base receptacle 84. When the fragrance cartridge 24 is installed in the receptacle 36, the left shaped cartridge member 60 can push the contact finger 89 inwardly which can close a contact (e.g., operate the electrical switch) thus connecting the electrical connector 29 with the heating element 38 to facilitate heating of the fragrance cartridge 24 with the heating element 38. When the fragrance cartridge 24 is not installed in the receptacle 36, the contact finger 89 is not operated and the heating element 38 is disconnected with the electrical connector 29 (e.g., powered). Thus, when the base unit 22 is plugged into a power source (e.g., a computer), the base unit 22 does not waste power by operating the heating element 38 unless the fragrance cartridge 24 is installed properly. Furthermore, since the heat from the heating element 38 is more effectively dissipated when the fragrance cartridge 24 is properly installed within the receptacle 36, the air freshener device 20 might be less prone to overheating and malfunction.

It will be appreciated that the base unit 22 can include any of a variety of suitable alternative electrical switch arrangements. For example, in another embodiment, an electrical switch can comprise a reed-type shutoff switch which can be selectively actuated with a magnetic field. In such an embodiment, a magnet can be associated with one of the shaped cartridge members (e.g., 60, 62, 64, 66, 68) and the reed-type shutoff switch can be associated with one of the shaped base receptacles (e.g., 84, 86, 88). When the fragrance cartridge 24 is not installed in the receptacle 36, a magnetic field is not received by the reed-type shutoff switch and the heating element 38 is not connected with the electrical connector 29. However, once the fragrance cartridge 24 is installed in the receptacle 36, one of the shaped cartridge members (e.g., 60, 62, 64, 66, 68) can impart a magnetic field to the reed-type shutoff switch to connect the electrical connector 29 and heating element 38 together and facilitate powering of the heating element 38.

In another example, an electrical switch can comprise a contactor-type shutoff switch. In such an example, the base unit 22 can include a pair of electrical contact pads (not shown) that are located opposite from one another within one of the shaped base receptacles (e.g., 84, 86, 88). One electrical contact pad can be in electrical communication with the electrical connector 29 and the other electrical contact pad can be in electrical communication with the heating element 38. One of the shaped cartridge members (e.g., 60, 62, 64, 66, 68) can include a pair of conductive fingers (not shown) that is in electrical communication with each other (e.g., shorted together). When the fragrance cartridge 24 is not installed in the receptacle 36, the circuit between the heating element 38 and the electrical connector 29 is open such that the heating element 38 is not connected with the electrical connector 29. However, when the fragrance cartridge 24 is installed in the receptacle 36, each conductive finger of the shaped cartridge member contacts the electrical contact pads to complete a circuit between the electrical connector 29 and the heating element 38 to facilitate activation of the heating element 38.

When the heating element 38 is installed in the fragrance cartridge 24 (e.g., with the electrical switch operated), activation of the heating element 38 can be controlled. In one embodiment, the heating element 38 can be activated once the fragrance cartridge 24 is installed in the receptacle and can remain activated until the fragrance cartridge 24 is removed from the receptacle 36 or the air freshener device 20 is unplugged from its power source. In another embodiment, a timing circuit (not shown) can selectively activate and deactivate the heating element 38 while the fragrance cartridge 24 is installed in the receptacle 36. In such an embodiment, the timing circuit can be configured to repeatedly activate (e.g., power) and deactivate (e.g., depower) the heating element 38 according to a predetermined timing arrangement. In particular, once the heating element 38 is activated, the timing circuit can await passage of a predefined period of time before deactivating the heating element 38. Consequently, once the heating element 38 has been deactivated for predetermined period of time, the timing circuit can then reactivate the heating element 38.

In yet another embodiment, when the air freshener device 20 is powered through the USB port of a computer (not shown), the computer can control operation of the air freshener device 20. Software can be configured to facilitate selective activation and deactivation of the heating element 38 at certain intervals. For example, the computer can activate and deactivate the heating element 38 every 30 minutes. An indicator (such as a light 90 as illustrated in FIGS. 1 and 5) can indicate to a user when the heating element 38 is activated or deactivated. When the air freshener device 20 is connected with the computer USB port, the computer can identify the air freshener device 20 and activate the heating element 38. Once the heating element 38 has been in operation for about 30 minutes, the computer can deactivate the heating element 38 for 30 minutes and then subsequently reactivate the heating element 38. It will be appreciated that the intervals can be predefined in the program or can alternatively be selectively programmed by a user (e.g., through use of a graphical user interface (GUI)). It will be appreciated that oftentimes, a scent produced by a fragrance device can become less effective (e.g., to a user) due to the effects of anosmia. The heating element 38 can therefore be selectively and automatically operated to overcome the effects of anosmia.

It will be appreciated that, when the air freshener device 20 is powered through a computer's USB port, software can be configured to achieve any of a variety of additional or alternative features. For example, software can be configured to facilitate control of the intensity of the scent provided from the air freshener device 20, such as through use of a GUI. Therefore, if the air freshener device 20 is not producing a strong enough scent for a user, the user can reprogram the intensity of the air freshener device to produce a more effective scent. In another example, the software can be configured to implement predetermined olfactory therapy routines such as might be provided in an aromatherapy session or other similar environment.

In one embodiment, as illustrated in FIGS. 1 and 5, the base unit 22 can include an on/off switch 92 that facilitates selective manual operation of the air freshener device 20. In one embodiment, the on/off switch 92 can be provided as a manual override for an automatic control scheme (e.g., a timing circuit, software control). However, in other embodiments, the on/off switch 92 can be provided in lieu of an automatic control scheme such that the on/off switch 92 is the only means to selectively operate the air freshener device 20.

The left and right shaped cartridge members 60, 68, the three central shaped cartridge members 62, 64, 66, and the left and right shaped cartridge receptacles 72, 74 are shown to cooperate to define three x-shaped features. It will be appreciated that any combination of shaped members and/or shaped receptacles can be provided in any of a variety of suitable arrangements upon a fragrance cartridge and/or a base. For example, a cartridge can include only one shaped member that is configured to actuate an electrical switch disposed with a shaped base receptacle.

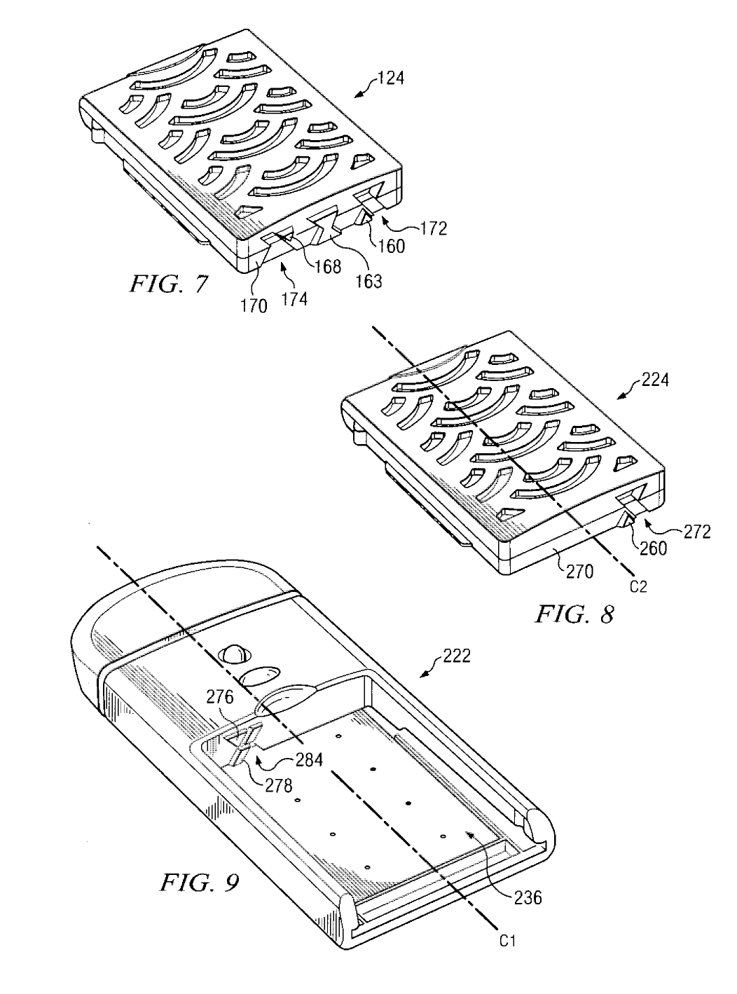

FIG. 7 illustrates a fragrance cartridge 124 according to another embodiment. The fragrance cartridge 124 illustrated in FIG. 7 can be similar in many respects to the fragrance cartridge 24 shown in FIG. 1. For example, the fragrance cartridge 124 can include a left shaped cartridge member 160 and a right shaped cartridge member 168 that each protrude from an end wall 170. The fragrance cartridge 124 can also include left and right shaped cartridge receptacles 172, 174 that are defined by the end wall 170. The fragrance cartridge 124, however, includes one central x-shaped cartridge member 163 that is substantially x-shaped.

FIGS. 8 and 9 illustrate an air freshener device 220 according to another embodiment. The air freshener device 220 illustrated in FIGS. 8 and 9 can be similar in many respects to the air freshener device 20 shown in FIG. 1. For example, the air freshener device 220 can include a fragrance cartridge 224 (FIG. 8) and a base unit 222 (FIG. 9). However, the fragrance cartridge 224 can include only a left shaped cartridge member 260 and a left shaped cartridge receptacle 272 defined by an end wall 270. The base unit 222 can include only two left shaped base members 276, 278 and a left shaped base receptacle 284. The base unit 222 and the fragrance cartridge 224 can define respective centerlines C1 and C2. The two left shaped base members 276, 278 and the left shaped base receptacle 284 can be offset from the centerline C1 and the left shaped cartridge member 260 and left shaped cartridge receptacle 272 can be offset from the centerline C2. The fragrance cartridge 224 can accordingly be inserted into a receptacle 236 in only one orientation and direction that will permit the shaped members and shaped receptacles to properly mate.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent devices by those of ordinary skill in the art. Rather it is hereby intended the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is clamed is:

1. An air freshener device comprising:
a base unit comprising:
a first end comprising an electrical connector; and
a second end comprising:
a floor portion;
a heating element coupled with the floor portion and electrically coupled with the electrical connector;
a pair of interior sidewalls extending substantially vertically from the floor portion and on opposite sides of the heating element;
an interior end wall extending substantially vertically from the floor portion and extending between the pair of interior sidewalls, the interior end wall and the pair of interior sidewalls cooperating to at least partially define a receptacle; and
a first shaped feature associated with one of the floor portion, the interior end wall, and one of the pair of interior sidewalls; and
a fragrance cartridge at least partially disposed within the receptacle and overlying the heating element, the fragrance cartridge comprising:
an absorbent pad configured to selectively retain a fragrance composition;
a shell body that substantially encompasses and supports the absorbent pad and comprises a shell wall; and
a second shaped feature associated with the shell body and configured for mated interaction with the first shaped feature of the base unit; and
wherein the fragrance cartridge is configured for selective installation within the receptacle.

2. The air freshener device of claim 1 wherein the first shaped feature comprises a shaped member that extends from the interior end wall and the second shaped feature comprises a shaped receptacle defined by the shell wall, the shaped member and the shaped receptacle being configured for mated interaction.

3. The air freshener device of claim 2 wherein the shaped member and the shaped receptacle are substantially x-shaped.

4. The air freshener device of claim 1 wherein the first shaped feature comprises a shaped receptacle defined by the interior end wall and the second shaped feature comprises a shaped member that extends from the shell wall, the shaped receptacle and the shaped member being configured for mated interaction.

5. The air freshener device of claim 1 wherein the base unit defines a first centerline that bisects the base unit into left and right sides and the fragrance cartridge defines a second centerline that bisects the fragrance cartridge into left and right sides, wherein the first and second shaped features are offset from the respective first and second centerlines.

6. The air freshener device of claim 1 wherein at least one of the interior sidewalls of the base unit defines a locking recess, the fragrance cartridge further comprises a locking protrusion, and the locking protrusion selectively engages the locking recess utilizing an interference-type fit to facilitate retention of the fragrance cartridge with respect to the base unit.

7. The air freshener device of claim 1 wherein the base unit further comprises an electrical switch in electrical communication with each of the electrical connector and the heating element, the electrical switch being operable to facilitate selective activation of the heating element.

8. The air freshener device of claim 7 wherein the electrical switch is associated with the first shaped feature, such that when the first shaped feature is mated with the second shaped feature, the electrical switch is operated and the heating element is controlled such that the heating element is activated.

9. The air freshener device of claim 7 wherein the electrical switch is associated with the first shaped feature, such that when the first shaped feature is mated with the second shaped feature, the electrical switch is operated and the heating element is controlled such that the heating element is selectively activated according to a predetermined timing arrangement.

10. The air freshener device of claim 1 wherein the shell body comprises a first shell portion and a second shell portion which are coupled together.

11. The air freshener device of claim 1 wherein the electrical connector comprises a USB plug.

12. The air freshener device of claim 1 wherein the absorbent pad comprises an absorbent material, the absorbent material comprising at least one of cardboard, recycled newspaper, wood pulp, cotton, porous plastic, and ceramic.

13. The air freshener device of claim 1 wherein the first and second shaped features are arranged to permit installation of the fragrance cartridge within the receptacle in one of an upright position and an upside-down position.

14. The air freshener device of claim 13 wherein the base unit is configured to operate in different modes based upon a position of the fragrance cartridge within the receptacle.

15. A method for operating an air freshener device that comprises a base unit and a fragrance cartridge, the base unit defining a receptacle and including an electrical connector and a heating element electrically coupled with the electrical connector, wherein the fragrance cartridge is configured for selective installation into the receptacle, the method comprising:
connecting the electrical connector with a power source such that electrical power is transmitted to the electrical connector;
deactivating the heating element when the fragrance cartridge is not installed in the receptacle; and
controlling activation of the heating element when the fragrance cartridge is installed into the receptacle.

16. The method of claim 15 wherein controlling activation of the heating element comprises activating the heating element when the fragrance cartridge is installed in the receptacle.

17. The method of claim 15 wherein controlling activation of the heating element comprises selectively activating the heating element at predefined intervals.

18. The method of claim 17 wherein the electrical connector comprises a USB plug and the power source comprises a USB port of a computer, and wherein selectively activating the heating element at predefined intervals is achieved with a computer.

19. An air freshener device comprising:
a base unit comprising:
a front end comprising a USB plug;
a rear end comprising:
a floor portion;
a heating element coupled with the floor portion and electrically coupled with the USB plug;
a pair of interior sidewalls extending substantially vertically from the floor portion and on opposite sides of the heating element;
an interior end wall extending substantially vertically from the floor portion and extending between the pair of interior sidewalls, the interior end wall and the pair of interior sidewalls cooperating to at least partially define a receptacle; and
a shaped receptacle defined by the interior end wall; and
an electrical switch in electrical communication with each of the USB plug and the heating element, the electrical switch being associated with the shaped receptacle; and
a fragrance cartridge at least partially disposed within the receptacle and overlying the heating element, the fragrance cartridge comprising:
an absorbent pad configured to selectively retain a fragrance composition;
a shell body that substantially encompasses and supports the absorbent pad and comprises a shell wall; and
a shaped member that extends from the shell wall and is configured for mated interaction with the shaped receptacle of the base unit; wherein:
the fragrance cartridge is configured for selective installation within the receptacle;
the electrical switch is configured for operation when the shaped member is mated with the shaped receptacle; and
when the electrical switch is operated, the heating element is selectively activated according to a predetermined timing arrangement.

20. The air freshener device of claim 19 wherein at least one of the interior sidewalls of the base unit defines a locking recess, the fragrance cartridge further comprises a locking protrusion, and the locking protrusion selectively engages the locking recess utilizing an interference-type fit to facilitate retention of the fragrance cartridge with respect to the base unit.
